(19) United States
(12) Patent Application Publication
(10) Pub. No.: US 2026/0041937 A1
(43) Pub. Date: Feb. 12, 2026

(54) DYNAMIC VOLUME REGISTRATION FOR IMAGE-GUIDED RADIOTHERAPY

(71) Applicant: ACCURAY LLC, Madison, WI (US)

(72) Inventors: Jacob D. Shea, Cross Plains, WI (US); Robert M. Pohlman, Deforest, WI (US)

(21) Appl. No.: 18/799,337

(22) Filed: Aug. 9, 2024

(51) Int. Cl. A61N 5/10

(52) U.S. Cl. CPC A61N 5/1067; A61N 5/1045; A61N 5/1049

(57) ABSTRACT

A method of radiation treatment delivery includes generating a three-dimensional (3D) reference image of a patient and generating a 3D image of a portion of the patient along a lengthwise axis. The method further includes performing a registration of the 3D image of the portion of the patient to a corresponding portion of the 3D reference image and controlling a radiation treatment beam based on the registration.

### TECHNICAL FIELD

The present disclosure relates to dynamic partial patient imaging for radiation delivery tracking and compensation.

### BACKGROUND

In radiation treatment, a radiation delivery system may utilize imaging for patient alignment, motion tracking, and verification of dose delivered to the subject. 3D volumetric images are useful for visualizing and locating the relative positions of tumors (i.e., the target of radiotherapy treatments) and surrounding organs. 3D volumetric image registrations can be used to align a radiation treatment plan with a position of the patient.

### BRIEF DESCRIPTION OF THE DRAWINGS

The present disclosure will be understood more fully from the detailed description given below and from the accompanying drawings of various implementations of the disclosure.

FIG. 1A illustrates a helical radiation delivery system, in accordance with embodiments described herein.

FIG. 1B illustrates a robotic radiation treatment system that may be used in accordance with embodiments described herein.

FIG. 1C illustrates a C-arm gantry-based radiation treatment system, in accordance with embodiments described herein.

FIG. 2 is an illustration of an example of radiation treatment planning and delivery system, in accordance with embodiments of the disclosure.

FIG. 3 is an illustration of an example of a volumetric imaging system of a radiation delivery system to generate partial volume images of a patient, in accordance with embodiments of the disclosure.

FIG. 4 is an illustration of an example of a patient volume imaged in multiple partial volumes along a lengthwise axis (Y-axis) for partial volume registration during radiation treatment, in accordance with embodiments of the disclosure.

FIG. 5 depicts a flow diagram of a method of partial volume image registration, in accordance with embodiments of the disclosure.

FIG. 6 depicts a flow diagram of another example method of partial volume image registration, in accordance with embodiments of the disclosure.

FIG. 7 depicts a flow diagram of an example method of performing radiation treatment using partial volume image registration, in accordance with embodiments of the disclosure.

FIG. 8 is a block diagram of an example computing device that may perform one or more of the operations described herein, in accordance with some embodiments.

### DETAILED DESCRIPTION

Described herein are embodiments for radiation treatment delivery using partial volumetric image registration. A radiation delivery system may include one or more radiation sources that generate a therapeutic radiation beam to deliver a therapeutic dose of radiation to a target, such as a tumor, and one or more imaging radiation beams which are used to acquire imaging data associated with the target.

In a conventional radiation delivery system, patient treatment begins with patient setup, followed by registration of patient position (determined from an image taken after setup) to a reference or treatment planning image (e.g., a three-dimensional CT or other scan), followed by the treatment of the patient using a predetermined treatment plan. Accordingly, in such conventional systems a full body rigid registration is performed prior to beginning treatment to align the treatment beam based on the treatment plan. The use of full body rigid registration, however, does not account for change of patient position, movement of organs, or any other sources of movement or error introduced between the time of registration and completion of the treatment. Conventional systems do not provide or incorporate feedback during treatment to make updates to the three-dimensional registration accounting for sources of error. Additionally, using full-body rigid registrations may provide for an overfit to a particular location in the body, the treatment of which may therefore suffer treatment delivery error which may further be introduced to other portions of the volume. Similarly, using full body rigid registrations may introduce errors due to non-rigid contortion of the anatomy at the time of treatment relative to the reference image. For example, variations in the curvature of the spine of the may result in registration error (e.g., due to overfitting a particular location of the body). In other words, by fitting the registration image to one section or area of the body correctly, the rest of the registration may include errors from non-rigid variations of the patient position and anatomy.

Aspects of the disclosure may remedy the above and other deficiencies by performing dynamic partial volume registrations during treatment delivery. During radiation treatment, a treatment delivery system may divide the length of reference image of a patient, or section of a patient, into multiple volumetric slices, or partial volumes. The treatment delivery system may generate an imaging beam to generate a three-dimensional (3D) image of the patient over one of the partial volumes (e.g., slices). The treatment delivery system may then register the partial volume to the reference image (e.g., to a portion of the reference image corresponding to the partial volume). In some embodiments, the treatment delivery system may generate the image during treatment prior to delivering a treatment beam to the partial volume. Accordingly, as the treatment progresses, a 3D volumetric image of the patient may be generated for a partial volume of the patient and a new local registration of the partial volume performed prior to the treatment beam reaching the partial volume. This process may be performed iteratively, or continuously, as radiation treatment is performed such that a new partial registration is performed at each slice or partial volume of the patient before treatment is performed on that corresponding partial volume.

In some embodiments, the partial imaging and partial image registration is performed immediately before the treatment beam (e.g., the imaging beam leads the treatment beam). In some embodiments, a complete partial volume is imaged and registered (e.g., stepwise) prior to applying the treatment beam to the partial volume. In some embodiments, the partial image registration includes determining a translation between the 3D image of a partial volume and the corresponding portion of the 3D reference image to determine how far away the target or organs at risk are from where they are expected to be according to the treatment plan and reference image. The registration is thus updated to capture the translation between the partial volumes throughout the full extent of the treatment delivery. The resulting registration of the patient is therefore a function of the position along the lengthwise axis of the patient.

Dynamic partial volume image registration during radiation therapy provides for an improved radiation delivery system with more accurate therapeutic dose delivery to the target. The dynamic registration reduces the time between registration and dose delivery, thus reducing the time in which errors can be introduced into the patient and target position. Additionally, the near real-time registration during treatment delivery provides for improved data collection for confirming or analyzing dosage and variations in dosage. Further, the dynamic registration may be used to perform real-time compensation of the treatment beam based on more accurate target positioning. In some embodiments, the partial volume image registrations may be used to modify therapeutic radiation delivery to compensate for the motion or other visible change of the target, ensuring that the appropriate therapeutic dose is accurately delivered to the target and unnecessary radiation exposure to non-target areas, such as sensitive organs, is minimized. For example, processing logic may use the partial image registrations to adaptively modify, during treatment, a treatment delivery that is to be applied to the patient. In another example, the processing logic may adjust the treatment beam or target alignment to compensate for target movement, setup error, misalignment, or any other sources of error in target position relative to the treatment plan.

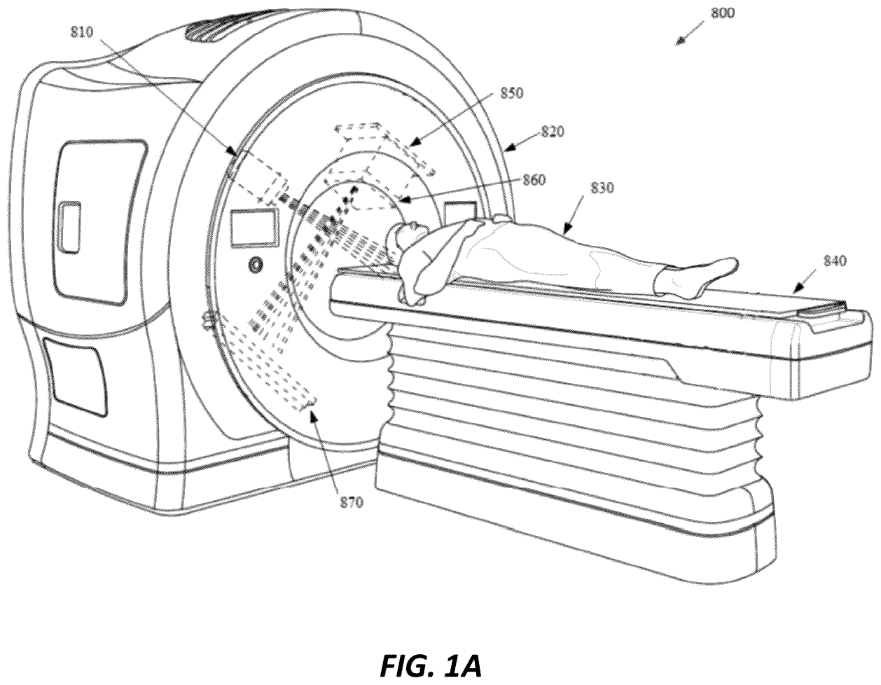

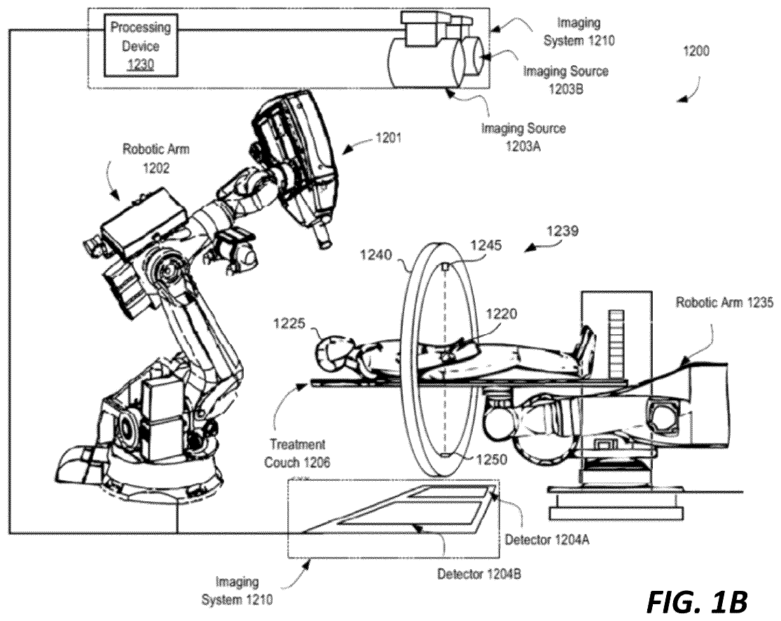

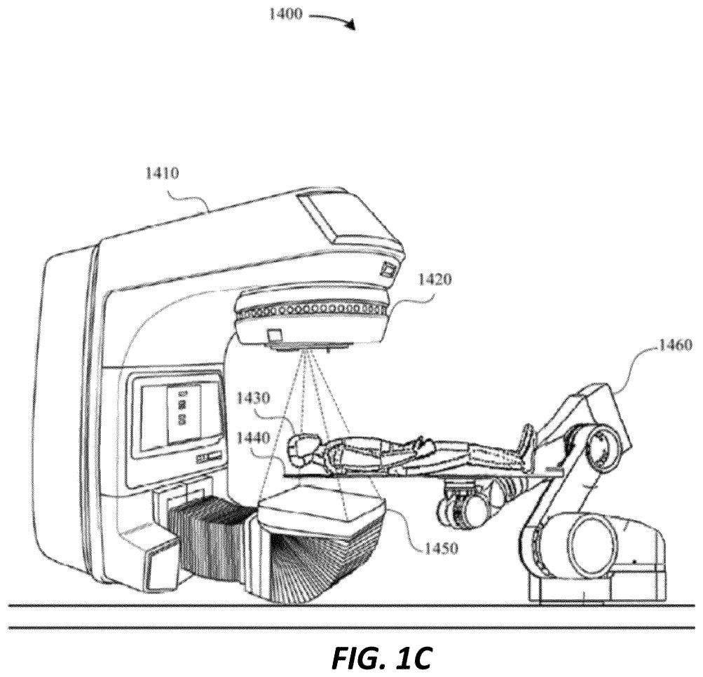

FIG. 1A illustrates a helical radiation delivery system 800 in accordance with embodiments of the present disclosure. The helical radiation delivery system 800 may include a linear accelerator (LINAC) 850 mounted to a ring gantry 820. The LINAC 850 may be used to generate a radiation beam (i.e., treatment beam) by directing an electron beam towards an x-ray emitting target. The treatment beam may deliver radiation to a target region (i.e., a tumor). The treatment system further includes a multileaf collimator (MLC) 860 coupled with the distal end of the LINAC 850. The MLC includes a housing that houses multiple leaves that are movable to adjust an aperture of the MLC to enable shaping of the treatment beam. In embodiments, the MLC 860 may be a binary MLC that includes a plurality of leaves arranged in two opposing banks, where the leaves of the two opposing banks are interdigitated with one another and can be opened or closed to form an aperture. In some embodiments, the MLC 860 may be an electromagnetically-actuated MLC. In embodiments, MLC 860 may be any other type of MLC. The ring gantry 820 has a toroidal shape in which the patient 830 extends through a bore of the ring/toroid and the LINAC 850 is mounted on the perimeter of the ring and rotates about the axis passing through the center to irradiate a target region with beams delivered from one or more angles around the patient. During treatment, the patient 830 may be simultaneously moved through the bore of the gantry on a treatment couch 840.

The helical radiation delivery system 800 includes an imaging system, comprising the LINAC 850 as an imaging source and an x-ray detector 870. The LINAC 850 may be used to generate a mega-voltage x-ray image (MVCT) of a region of interest (ROI) of patient 830 by directing a sequence of x-ray beams at the ROI which are incident on the x-ray detector 870 opposite the LINAC 850 to image the patient 830 for setup and generate pre-treatment images. In one embodiment, the helical radiation delivery system 800 may also include a secondary imaging system consisting of a kV imaging source 810 mounted orthogonally relative to the LINAC 850 (e.g., separated by 90 degrees) on the ring gantry 820 and may be aligned to project an imaging x-ray beam at a target region and to illuminate an imaging plane of a detector after passing through the patient 130. In some embodiments, the kV imaging source 810 may be mounted at any physically available angle relative to the LINAC 850.

FIG. 1B illustrates a radiation treatment system 1200 that may be used in accordance with alternative embodiments described herein. As shown, FIG. 1B illustrates a configuration of a radiation treatment system 1200. In the illustrated embodiments, the radiation treatment system 1200 includes a linear accelerator (LINAC) 1201 that acts as a radiation treatment source and an MLC 1205 coupled with the distal end of the LINAC 1201 to shape the treatment beam. In one embodiment, the LINAC 1201 is mounted on the end of a robotic arm 1202 having multiple (e.g., 5 or more) degrees of freedom in order to position the LINAC 1201 to irradiate a pathological anatomy (e.g., target) with beams delivered from many angles, in many planes, in an operating volume around a patient. Treatment may involve beam paths with a single isocenter, multiple isocenters, or with a non-isocentric approach.

LINAC 1201 may be positioned at multiple different nodes (predefined positions at which the LINAC 1201 is stopped and radiation may be delivered) during treatment by moving the robotic arm 1202. At the nodes, the LINAC 1201 can deliver one or more radiation treatment beams to a target, where the radiation beam shape is determined by the leaf positions in the MLC 1205. The nodes may be arranged in an approximately spherical distribution about a patient. The particular number of nodes and the number of treatment beams applied at each node may vary as a function of the location and type of pathological anatomy to be treated.

In another embodiment, the robotic arm 1202 and LINAC 1201 at its end may be in continuous motion between nodes while radiation is being delivered. The radiation beam shape and 2-D intensity map is determined by rapid motion of the leaves in the MLC 1205 during the continuous motion of the LINAC 1201.

The radiation treatment system 1200 includes an imaging system 1210 having a processing device 1230 connected with x-ray sources 1203A and 1203B (i.e., imaging sources) and fixed x-ray detectors 1204A and 1204B. Alternatively, the x-ray sources 1203A, 1203B and/or x-ray detectors 1204A, 1204B may be mobile, in which case they may be repositioned to maintain alignment with the target, or alternatively to image the target from different orientations or to acquire many x-ray images and reconstruct a three-dimensional (3D) cone-beam CT. In one embodiment, the x-ray sources are not point sources, but rather x-ray source arrays, as would be appreciated by the skilled artisan. In one embodiment, LINAC 1201 serves as an imaging source, where the LINAC power level is reduced to acceptable levels for imaging.

Imaging system 1210 may perform computed tomography (CT) such as cone beam CT, helical megavoltage computed tomography (MVCT), or kilovoltage computed tomography (CT), and images generated by imaging system 1210 may be two-dimensional (2D) or three-dimensional (3D). The two x-ray sources 1203A and 1203B may be mounted in fixed positions on the ceiling of an operating room and may be aligned to project x-ray imaging beams from two different angular positions (e.g., separated by 90 degrees) to intersect at a machine isocenter (referred to herein as a treatment center, which provides a reference point for positioning the patient on a treatment couch 1206 during treatment) and to illuminate imaging planes of respective detectors 1204A and 1204B after passing through the patient. In one embodiment, imaging system 1210 provides stereoscopic imaging of a target and the surrounding volume of interest (VOI). In other embodiments, imaging system 1210 may include more or less than two x-ray sources and more or less than two detectors, and any of the detectors may be movable rather than fixed. In yet other embodiments, the positions of the x-ray sources and the detectors may be interchanged. In embodiments, the imaging data acquired from the fixed angles of by imaging system 1210 may be combined with prior images, such as a planning image or pre-treatment CT image. The imaging data acquired from the fixed angles acquired during therapeutic radiation beam delivery may be used to deform the prior images into a volumetric image representing the patient at the time of therapeutic radiation beam delivery. Detectors 1204A and 1204B may be fabricated from a scintillating material that converts the x-rays to visible light (e.g., amorphous silicon), and an array of CMOS (complementary metal oxide silicon) or CCD (charge-coupled device) imaging cells that convert the light to a digital image that can be compared with a reference image during an image registration process that transforms a coordinate system of the digital image to a coordinate system of the reference image, as is well known to the skilled artisan. The reference image may be, for example, a digitally reconstructed radiograph (DRR), which is a virtual x-ray image that is generated from a 3D CT image based on simulating the x-ray image formation process by casting rays through the CT image.

In one embodiment, IGRT delivery system 1200 also includes a secondary imaging system 1239. Imaging system 1239 may be a Cone Beam Computed Tomography (CBCT) imaging system. Alternatively, other types of volumetric imaging systems may be used. The secondary imaging system 1239 includes a rotatable gantry 1240 (e.g., a ring) attached to an arm and rail system (not shown) that move the rotatable gantry 1240 along one or more axes (e.g., along an axis that extends from a head to a foot of the treatment couch 1206. An imaging source 1245 and a detector 1250 are mounted to the rotatable gantry 1240. The rotatable gantry 1240 may rotate 360 degrees about the axis that extends from the head to the foot of the treatment couch. Accordingly, the imaging source 1245 and detector 1250 may be positioned at numerous different angles. In one embodiment, the imaging source 1245 is an x-ray source and the detector 1250 is an x-ray detector. In one embodiment, the secondary imaging system 1239 includes two rings that are separately rotatable. The imaging source 1245 may be mounted to a first ring and the detector 1250 may be mounted to a second ring. In one embodiment, the rotatable gantry 1240 rests at a foot of the treatment couch during therapeutic radiation beam delivery to avoid collisions with the robotic arm 1202.

As shown in FIG. 1B, the image-guided radiation treatment system 1200 may further be associated with a treatment delivery workstation 150. The treatment delivery workstation may be remotely located from the radiation treatment system 1200 in a different room than the treatment room in which the radiation treatment system 1200 and patient are located. The treatment delivery workstation 150 may include a processing device (which may be processing device 1230 or another processing device) and memory that modify a treatment delivery to the patient 1225 based on a detection of a target motion that is based on one or more image registrations, as described herein.

FIG. 1C illustrates a C-arm radiation delivery system 1400. In one embodiment, in the C-arm system 1400 the beam energy of a LINAC may be adjusted during treatment and may allow the LINAC to be used for both x-ray imaging and radiation treatment. In another embodiment, the system 1400 may include an onboard kV imaging system to generate x-ray images and a separate LINAC to generate the higher energy therapeutic radiation beams. The system 1400 includes a C-arm gantry 1410, a LINAC 1420, an MLC 1470 coupled with the distal end of the LINAC 1420 to shape the beam, and a portal imaging detector 1450. The C-arm gantry 1410 may be rotated to an angle corresponding to a selected projection and used to acquire an x-ray image of a VOI of a patient 1430 on a treatment couch 1440. In embodiments that include a portal imaging system, the LINAC 1420 may generate an x-ray beam that passes through the target of the patient 1430 and are incident on the portal imaging detector 1450, creating an x-ray image of the target. After the x-ray image of the target has been generated, the beam energy of the LINAC 1420 may be increased so the LINAC 1420 may generate a radiation beam to treat a target region of the patient 1430. In another embodiment, the kV imaging system may generate an x-ray beam that passes through the target of the patient 1430, creating an x-ray image of the target. In some embodiments, the portal imaging system may acquire portal images during the delivery of a treatment. The portal imaging detector 1450 may measure the exit radiation fluence after the beam passes through the patient 1430. This may enable internal or external fiducials or pieces of anatomy (e.g., a tumor or bone) to be localized within the portal images.

Alternatively, the kV imaging source or portal imager and methods of operations described herein may be used with yet other types of gantry-based systems. In some gantry-based systems, the gantry rotates the kV imaging source and LINAC around an axis passing through the isocenter. In other embodiments, the kV imaging source and the LINAC may rotate independent of each other, while being able to image and deliver therapeutic radiation simultaneously to the same volume of interest. Gantry-based systems include ring gantries having generally toroidal shapes in which the patient's body extends through the bore of the ring/toroid, and the kV imaging source and LINAC are mounted on the perimeter of the ring and rotates about the axis passing through the isocenter. Gantry-based systems may further include C-arm gantries, in which the kV imaging source and LINAC are mounted, in a cantilever-like manner, over and rotates about the axis passing through the isocenter. In another embodiment, the kV imaging source and LINAC may be used in a robotic arm-based system, which includes a robotic arm to which the kV imaging source and LINAC are mounted as discussed above. Aspects of the present disclosure may further be used in other such systems such as a gantry-based LINAC system, static imaging systems associated with radiation therapy and radiosurgery, proton therapy systems using an integrated image guidance, interventional radiology and intraoperative x-ray imaging systems, etc.

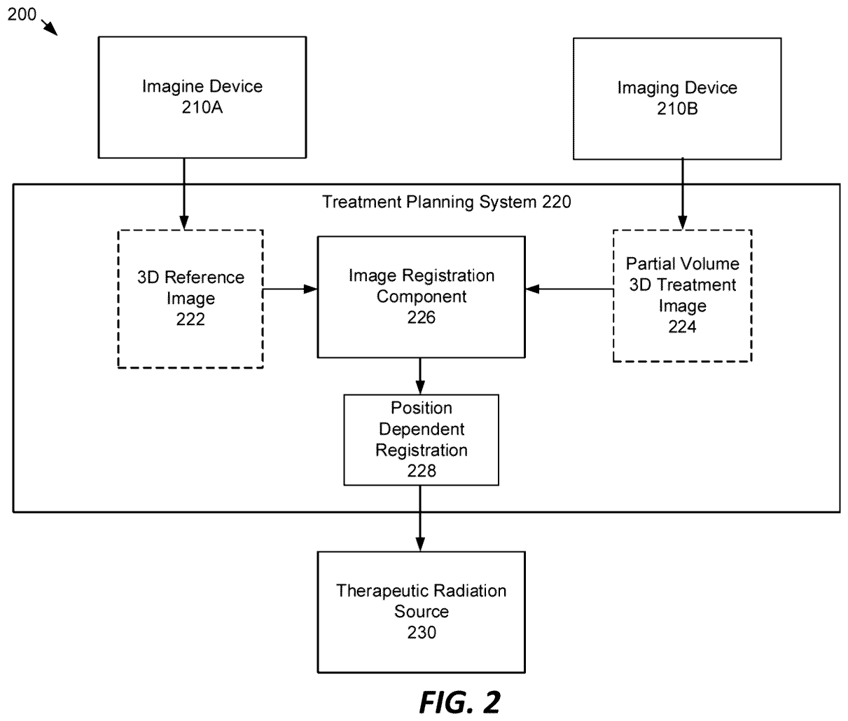

FIG. 2 is an illustration of an example of radiation delivery system 200 for generating a position dependent registration using partial volume registrations, in accordance with embodiments of the disclosure. In embodiments, the radiation delivery system 200 may correspond to, and include the components of, one of the radiation delivery systems previously described at FIGS. 1A-C. Radiation delivery system 200 may include one or more imaging devices 210A-B for generating 2D images or 3D images of a patient or areas of interest of a patient. For example, imaging device 210A may generate a 3D reference image 222 of a patient prior to treatment of the patient. In some embodiments, a treatment planning system 220 may use the 3D reference image 222 to generate a treatment plan for the patient. For example, the 3D reference image 222 may include one or more target areas within the patient that are to be treated with a particular dosage of radiation therapy. The treatment planning system 220 may thus identify the one or more target areas from the 3D reference image 222 and determine treatment doses to be provided to the one or more target areas from various angles. A

[symbol]

In some embodiments, imaging device 210B may be an imaging device that provides an imaging beam (e.g., imaging radiation beam) during treatment of the patient to image portions of the patient during treatment, such as for target tracking or dynamic partial image registration. For example, the imaging device 210B may generate a partial volume 3D treatment image 224 representing a slice (e.g., volume along a lengthwise axis) of the patient. The treatment planning system 220 may further include an image registration component 226 for performing an partial volume image registration for each partial volume 3D treatment image 224. For example, the image registration component 226 may register the partial volume 3D treatment image 224 to a corresponding portion of the 3D reference image 222. In other words, the image registration component 226 performs a local 3D image registration of the partial volume 3D treatment image to the 3D reference image 222. The imaging device 210B may continuously or intermittently obtain or generate partial volume 3D treatment images 224 throughout treatment of the patient such that an overall registration during treatment is position dependent.

Accordingly, the image registration component 226 may continuously or intermittently perform partial 3D image registration throughout treatment to produce a position dependent registration 228. The position dependent registration 228 may thus provide translation between the various partial volume 3D treatment images 224 and the 3D reference image 222 based on where along the lengthwise axis of the patient that treatment is occurring. For example, a first translation may be associated with a first partial volume of the patient, a second translation may be associated with a second partial volume, a third translation may be associated with a third partial volume, and so forth.

In some embodiments, the treatment planning system 220 may then use the position dependent registration 228 to control a therapeutic radiation source 230. For example, the treatment planning system 220 may compensate the initial treatment plan to apply the expected dosage to the target. Because the partial registration is performed close in time to application of the treatment beam, the position dependent registration 228 can provide more accurate treatment beam compensation or control such that the proper dosage is provided to the target more accurately.

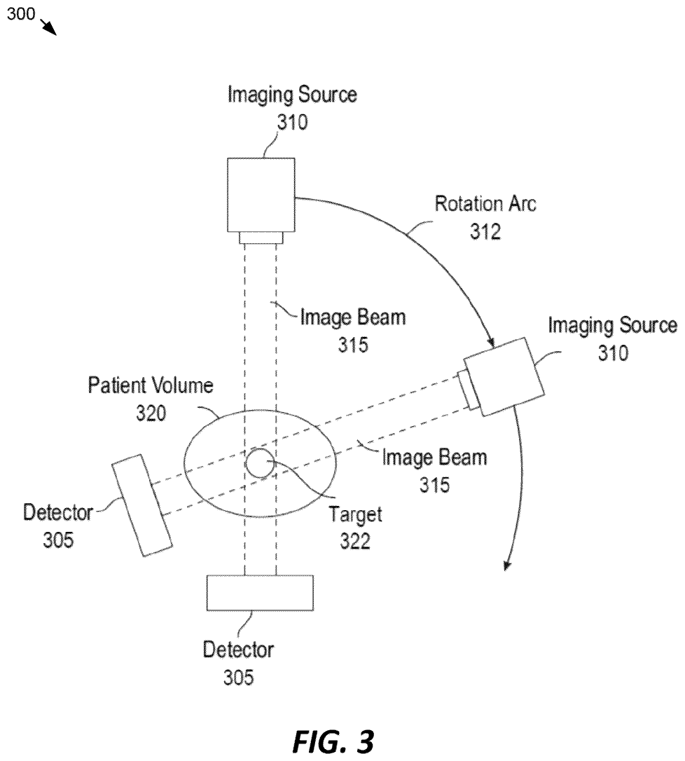

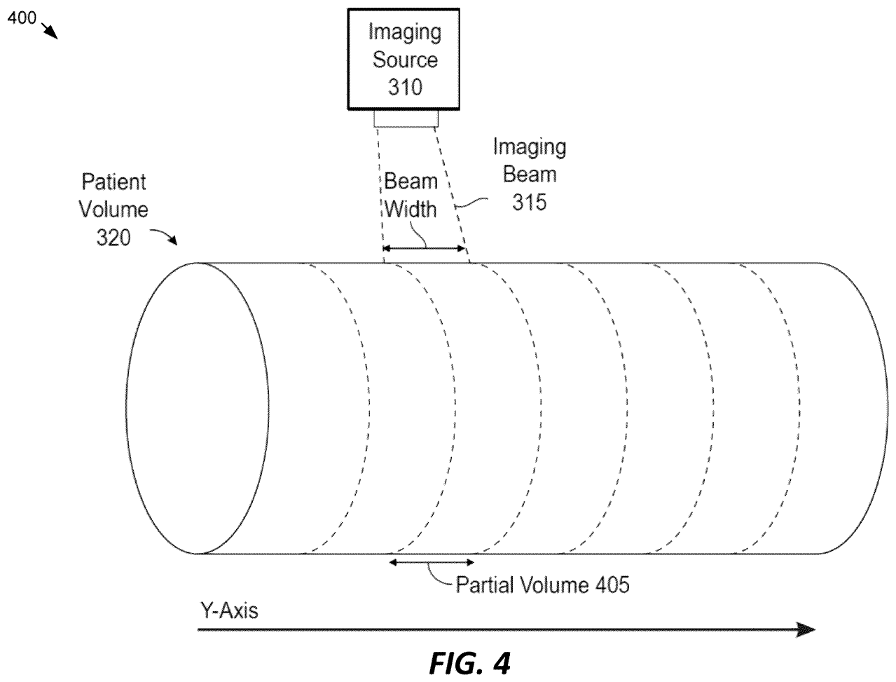

FIG. 3 is an illustration of an example of a radiation delivery and imaging system 300 for imaging partial 3D volumes of a patient, in accordance with embodiments of the disclosure. Radiation delivery and imaging system 300 may include similar components to the radiation delivery systems previously described at FIGS. 1A-2. The radiation delivery and imaging system 300 may utilize an imaging source 310 to generate an imaging beam 315. The imaging beam 315 may be used to produce, calculate, or otherwise generate a 3D image of a partial volume of a patient (e.g., patient volume 320). For example, the imaging source 310 may rotate along an arc of rotation 312 around the patient volume 320 and a target 322 of the patient volume. The imaging beam 315 may project through the patient volume 320 and target 322 to produce several images at a detector 305 as the imaging source 310 rotates about the patient. The produced images from the various angles around the patient may be used to produce a 3D image of a partial volume of the patient volume (e.g., a slice), as described in further detail with respect to FIG. 4. In some embodiments, the 3D image of the partial volume may be produced from a single rotation of the imaging source 310 around the patient or a combination of several rotations. For example, the imaging beam 315 may have a finite width (e.g., 5-30 cm) and thus may provide a single partial volume image of a corresponding finite width or of several rotations and thus a multiple of the image beam width.

In some embodiments, the radiation delivery and imaging system 300 may further include a therapeutic radiation source (not shown) to provide a radiation beam to irradiate the target 322 in the patient volume 320. The system 300 may perform a registration between the 3D image of a partial volume of the patient volume 320 and a treatment planning image, or reference image. For example, the imaging source 310 may lead the therapeutic radiation source during treatment such that a partial volume image is produced and a registration of the partial volume image is performed before the therapeutic radiation source performs the treatment on the target 322 within the partial volume. Thus, a local registration of 3D images taken during treatment can be performed dynamically during treatment of the patient to more accurately deliver a treatment dose to the target 322.

FIG. 4 illustrates an example imaging system 400 providing division of a patient volume into multiple partial volumes for dynamic 3D image registration during radiation treatment delivery, in accordance with embodiments of the disclosure. As depicted, system 400 includes an imaging source 310, which may be the same or similar to imaging source 310 of FIG. 3. Similarly, as described with respect to FIG. 3, imaging source 310 may rotation around a patient to produce images of a patient volume 320 which may be used for computing a 3D image of a partial volume 405 of the patient volume 320. In some embodiments, the imaging beam 315 produced by the imaging source 310 may have a particular width (e.g., beam width) that images a fraction of the patient volume 320 at one time. In some examples, a partial volume 405 that is imaged by a single rotation of the imaging source 310 (e.g., corresponding to the beam width) around the patient volume 320 is registered with a corresponding portion of a 3D reference image. Accordingly, a new registration may be performed for each partial volume 405 that is imaged during each rotation. In another example, images taken during multiple rotations of the imaging source 310 may be used as a partial volume for registration against the reference image. For example, two or more partial volumes 405 may be used together during a single registration with the reference image, which may reduce the granularity and accuracy of the registration but may reduce the computational costs associated with performing registration for each partial volume 405.

It should be noted, that although the partial volumes 405 are depicted in a stepwise arrangement, other embodiments, including a continuous helical pattern may also be used to image the patient volume 320. Additionally, a robotic imaging assembly (e.g., as depicted in FIG. 1B) may also be used for performing embodiments described herein.

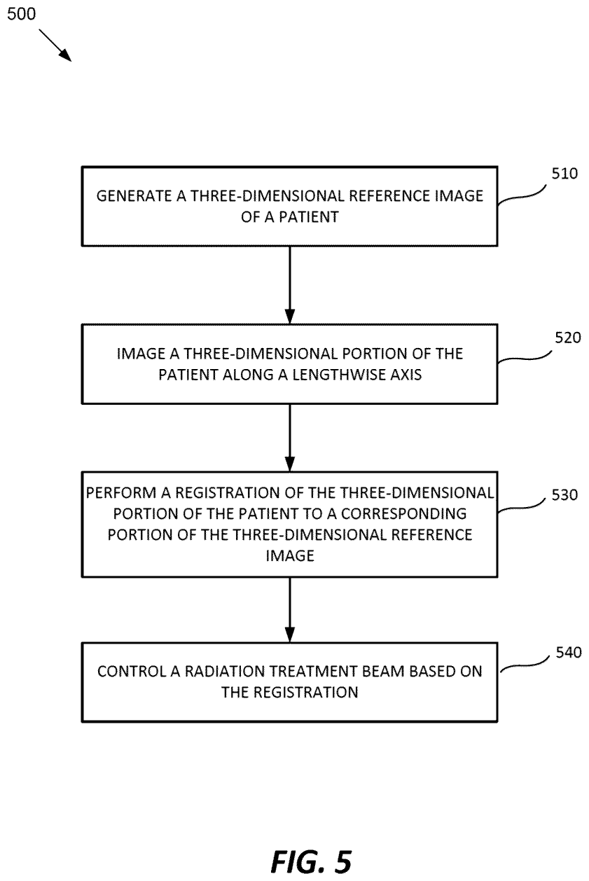

FIG. 5 depicts a flow diagram of a method 500 of dynamic 3D image registration using partial 3D volume images, in accordance with embodiments of the disclosure. Method 500 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In embodiments, various portions of method 500 may be performed by processing logic of a processing device of a radiation delivery system as previously described at FIGS. 1A-4.

With reference to FIG. 5, method 500 illustrates example functions used by various embodiments. Although specific function blocks (“blocks”) are disclosed in method 500, such blocks are examples. That is, embodiments are well suited to performing various other blocks or variations of the blocks recited in method 500. It is appreciated that the blocks in method 500 may be performed in an order different than presented, and that not all of the blocks in method 500 may be performed.

Method 500 begins at block 510, where the processing logic generates a three-dimensional reference image of a patient. The three-dimensional reference image may be a treatment planning image taken prior to treatment of the patient. For example, the 3D reference image may be a CT scan, MRI, or other 3D scan of the patient identifying target volumes and to generate a treatment plan to deliver a radiation dosage to the target volumes.

At block 520, the processing logic images a three-dimensional portion of the patient along a lengthwise axis. In some embodiments, the 3D image of the portion of the patient along the lengthwise axis may be a slice or partial volume of the patient that is imaged during radiation treatment delivery. For example, as the patient moves through the treatment field (e.g., on a treatment couch) or the treatment field moves along the patient, an imaging device may generate a 3D image of a partial volume or portion of the patient. In some embodiments, the imaging device may generate several two-dimensional images which are then combined to generate a 3D image of the imaged portion or slice of the patient. In some embodiments, the processing logic generates a plurality of 3D images for a plurality of partial volumes of the patient during treatment. For example, the plurality of 3D images and partial volumes may be co-planar volumes of the patient. Additionally, in some examples, the generation of the 3D images of the plurality of partial volumes may lead a treatment field of the radiation treatment beam.

At block 530, the processing logic performs a registration of the three-dimensional portion of the patient to a corresponding portion of the three-dimensional reference image. In some embodiments, the processing logic divides the reference image into a plurality of volumes along the lengthwise axis and locally registers the 3D image of the portion of the patient to the corresponding volume of the plurality of volumes along the lengthwise axis. In some embodiments, the lengthwise axis extends along the length of the patient. Accordingly, the 3D images are generated for different sections, volumes, or slices of the patient along the length of the patient. Thus, the registration of the plurality of 3D images of the partial volumes of the patient provides a spatially dependent registration of the patient to the 3D reference image.

At block 540, the processing logic controls a radiation treatment beam based on the registration. For example, in some embodiments the processing logic compensates the radiation treatment beam by controlling or adjusting a position of the leaves of a multi-leaf collimator or performing jaw shifts of the multi-leaf collimator based on the local registrations performed during the radiation treatment.

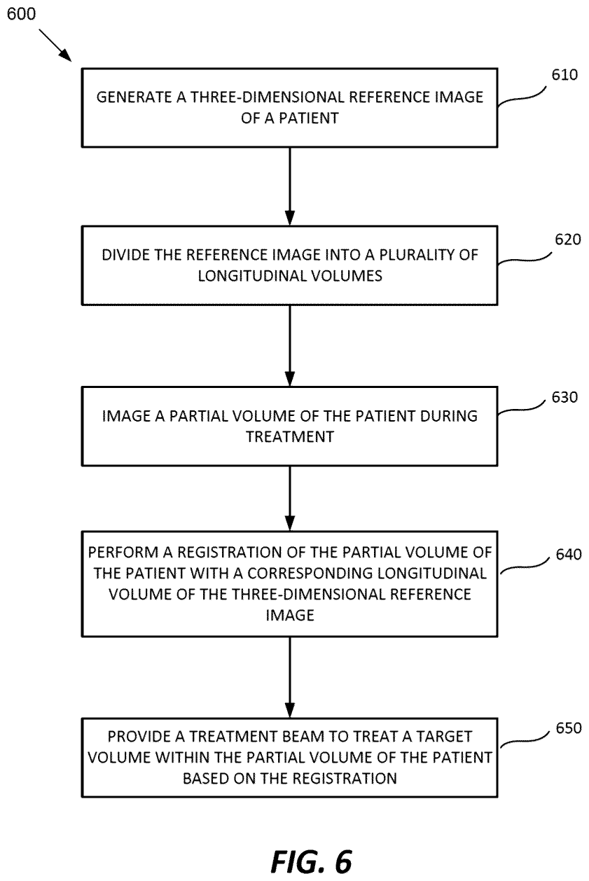

FIG. 6 depicts a flow diagram of a method 600 of dynamic 3D image registration using partial 3D volume images, in accordance with embodiments of the disclosure. Method 600 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In embodiments, various portions of method 600 may be performed by processing logic of a processing device of a radiation delivery system as previously described at FIGS. 1A-4.

With reference to FIG. 6, method 600 illustrates example functions used by various embodiments. Although specific function blocks (“blocks”) are disclosed in method 600, such blocks are examples. That is, embodiments are well suited to performing various other blocks or variations of the blocks recited in method 600. It is appreciated that the blocks in method 600 may be performed in an order different than presented, and that not all of the blocks in method 600 may be performed.

Method 600 begins at block 610, where processing logic generates a three-dimensional reference image of a patient. For example, prior to beginning radiation treatment of the patient, a 3D reference image or treatment planning image may be generated of the entire patient or of volumes of interest of the patient. The 3D reference image may be used for treatment planning, dosage determination (e.g., beam intensity and location from various angles) and so forth.

At block 620, the processing logic divides the 3D reference image into a plurality of longitudinal volumes. The longitudinal volumes may correspond to the width of an imaging beam used to image the patient during treatment. For example, the reference image may be divided into volumes that can be imaged during radiation treatment.

At block 630, the processing logic images a partial volume of the patient during treatment. The partial volume of the patient may include a cross-sectional volume of the patient imaged by an imaging source rotating around the patient. In some embodiments, multiple partial volume images may be generated as the radiation treatment progresses. For example, each partial volume image of the patient may be generated prior to the application of the radiation treatment to that volume of the patient (e.g., the imaging beam may lead the radiation treatment beam).

At block 640, the processing logic performs a registration of the partial volume of the patient with a corresponding longitudinal volume of the three-dimensional reference image. The registration may include a local translation, rotation, or both translation and rotation, between the 3D reference image and the partial volume image. By performing several registrations between the 3D reference image and a plurality of partial volume images, a position dependent registration, and thus translation, can be generated that provides a more accurate representation of the patient and target position during radiation treatment. Because each registration may be performed immediately before radiation delivery to the target within the corresponding portion, the time delay between registration and treatment delivery is minimized along with the error introduced by the delay.

At block 650, the processing logic provides a treatment beam to treat a target volume within the partial volume of the patient based on the registration. In some embodiments, a treatment beam is compensated or adjusted based on the registration (e.g., the most recent registration) to deliver the radiation dosage expected from the treatment plan.

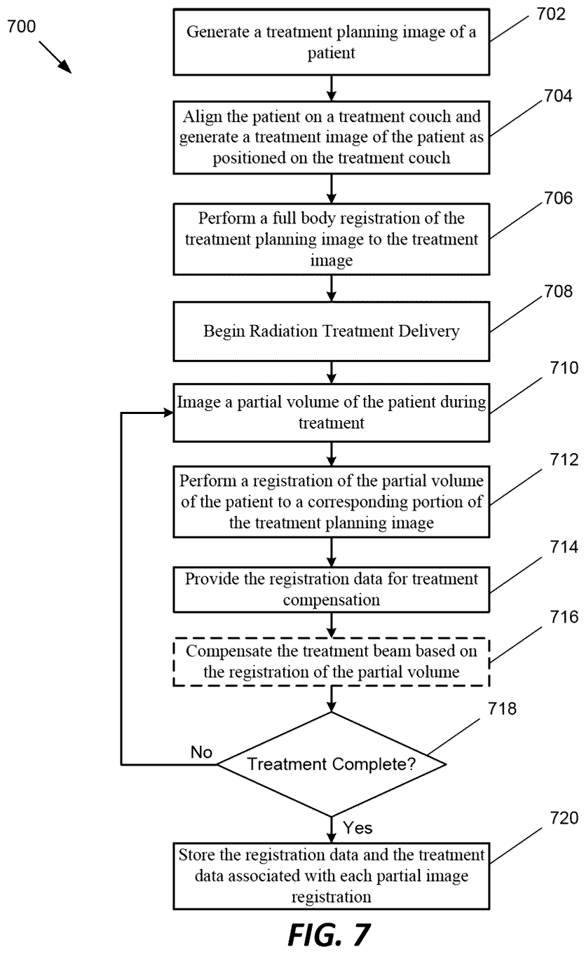

FIG. 7 depicts a flow diagram of a method 700 of radiation treatment compensation using dynamic partial volume image registration, in accordance with embodiments of the disclosure. Method 700 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In embodiments, various portions of method 700 may be performed by processing logic of a processing device of a radiation delivery system as previously described at FIGS. 1A-4.

With reference to FIG. 7, method 700 illustrates example functions used by various embodiments. Although specific function blocks (“blocks”) are disclosed in method 700, such blocks are examples. That is, embodiments are well suited to performing various other blocks or variations of the blocks recited in method 700. It is appreciated that the blocks in method 700 may be performed in an order different than presented, and that not all of the blocks in method 700 may be performed.

Method 700 begins at block 702, where processing logic generates a treatment planning image of a patient. In some embodiments, the treatment planning image may include a full body 3D image of the patient or a partial 3D image of the patient. The treatment planning image may be used to generate a treatment plan including radiation dosages to target areas of the patient. The treatment planning image may also be used as a reference to align the treatment beam during radiation treatment.

At block 704, the patient is aligned on a treatment couch and processing logic generates a setup image of the patient as positioned on the treatment couch. In some embodiments, this step may be referred to as patient setup where the patient is aligned on the treatment couch in order for radiation treatment to be performed. Additionally, the generation of the setup image may include generating a full 3D image of the patient on the treatment couch.

At block 706, the processing logic performs a full body registration of the treatment planning image to the setup image. In some embodiments, the full body registration of the treatment planning image to the setup image may include determining a single translation of all points (e.g., voxels) in the treatment planning image to the setup image. This initial full body registration may be optional and may be used to initialize alignment of radiation treatment delivery which may then be updated throughout the blocks 710-714 described below.

Alternatively, in some embodiments, rather than generating a setup image and aligning the patient based on the setup image, other means may be used for patient alignment. For example, rather than a setup image and initial registration, the patient may be aligned on the treatment couch using room lasers, surface imaging, or any other means for spatially aligning the patient.

At block 708, the processing logic begins radiation treatment delivery. At block 710, the processing logic images a partial volume of the patient during treatment. The partial volume may be a volume images by a source rotating around the patient. Accordingly, each volume may be an imaged slice of the patient. At block 712, the processing logic performs a registration of the partial volume of the patient to a corresponding portion of the treatment planning image. In some embodiments, the initial or first partial volume registration may be used to correct or reduce any alignment errors due to the initial setup of the patient. Similarly, processing logic may use the initial partial volume registration to adapt the treatment plan in real time to the current position of the patient during treatment. Each registration may be determined from the corresponding partial volume to the treatment planning image. The registration may include a translation between the partial volume and the treatment planning image and thus identifies how far away the target or organs at risk are from where they are expected to be based on the treatment planning image. In some embodiments, the registration is updated either continuously or incrementally over the full extent of treatment delivery. Accordingly, the partial volume registrations may be closer in time to treatment delivery to provide more accurate treatment beam delivery and to capture deformations that could not be captured by a single full body registration. The resulting registrations, or combination of registrations, are therefore a function of the position along the lengthwise axis of the patient.

At block 714, the processing logic provides the registration data for treatment compensation. In some embodiments, the local registration data can be used to update the treatment beam in real time. In some embodiments, the registration data may be provided to a user interface for a technician to act upon the information manually to allow the treatment to be manually allowed to continue, to be adjusted, or to be stopped (e.g., via a pause-resume cycle). For example, the technician may monitor patient alignment, treatment beam alignment, and other information provided by the dynamic registration data for each of the local registrations to determine if treatment should proceed or if an aspect of the radiation treatment delivery system or the patient positioning should be adjusted. The technician may then pause treatment, manually adjust or input adjustments of the system or the patient positioning, after which the technician may resume treatment of the patient from where the treatment was paused, or from another part of the treatment plan (e.g., skipping forward or starting from an earlier point in the treatment plan). Additionally, the technician may completely stop treatment if the technician identifies errors that cannot be corrected during treatment (e.g., incorrect treatment plan, major shifts in anatomy, etc.)

At block 716, the processing logic, optionally, compensates (e.g., automatically, without human intervention) the treatment beam based on the registration of the partial volume. At block 718, the processing logic determines if the treatment of the patient is complete. If the processing logic determines that treatment is not complete (e.g., additional partial volumes are to be treated), the process returns to block 710 to image another (e.g., the next adjacent) partial volume of the patient during treatment after which another image registration of the partial volume of the patient to the treatment planning image is performed, and so forth. Otherwise, if the processing logic determines that treatment is complete, the process proceeds to block 720, where the processing logic stores the registration data and the treatment data associated with each partial image registration. Accordingly, the stored registration and treatment data may allow examination of dosage and dosage variation after treatment is performed. Future treatments may thus be optimized based on the collected data.

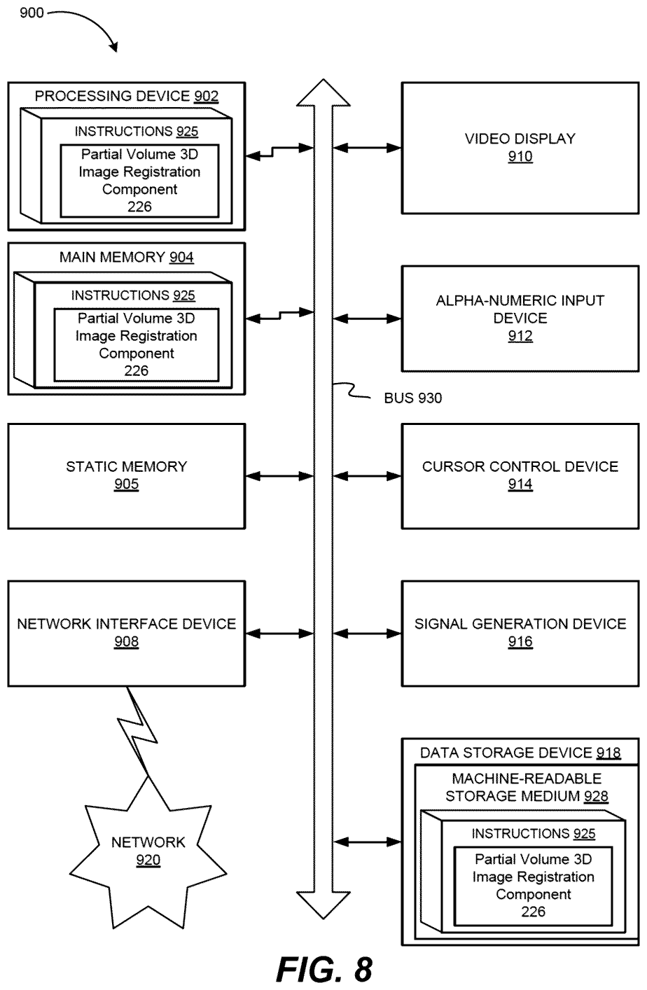

FIG. 8 is a block diagram of an example computing device 900 that may perform one or more of the operations described herein, in accordance with some embodiments. Computing device 900 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 900 may include a processing device (e.g., a general purpose processor, a PLD, etc.) 902, a main memory 904 (e.g., synchronous dynamic random access memory (DRAM), read-only memory (ROM)), a static memory 906 (e.g., flash memory and a data storage device 918), which may communicate with each other via a bus 930.

Processing device 902 may be provided by one or more general-purpose processing devices such as a microprocessor, central processing unit, or the like. In an illustrative example, processing device 902 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 902 may also comprise one or more special-purpose processing devices such as an application specific integrated circuit (ASIC), a field programmable gate array (FPGA), a digital signal processor (DSP), network processor, or the like. The processing device 902 may be configured to execute the operations described herein, in accordance with one or more aspects of the present disclosure, for performing the operations and steps discussed herein.

Computing device 900 may further include a network interface device 908 which may communicate with a network 920. The computing device 900 also may include a video display unit 910 (e.g., a liquid crystal display (LCD) or a cathode ray tube (CRT)), an alphanumeric input device 912 (e.g., a keyboard), a cursor control device 914 (e.g., a mouse) and an acoustic signal generation device 916 (e.g., a speaker). In one embodiment, video display unit 910, alphanumeric input device 912, and cursor control device 914 may be combined into a single component or device (e.g., an LCD touch screen).

Data storage device 918 may include a computer-readable storage medium 928 on which may be stored one or more sets of instructions that may include instructions 925 (e.g., instructions for partial volume 3D image registration component 226) for carrying out the operations described herein, in accordance with one or more aspects of the present disclosure. The instructions may also reside, completely or at least partially, within main memory 904 and/or within processing device 902 during execution thereof by computing device 900, main memory 904 and processing device 902 also constituting computer-readable media. The instructions may further be transmitted or received over a network 920 via network interface device 908.

While computer-readable storage medium 928 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

It should be noted that the methods and apparatus described herein are not limited to use only with medical imaging and treatment. In alternative implementations, the methods and apparatus herein may be used in applications outside of the medical technology field, such as industrial imaging and non-destructive testing of materials. In such applications, for example, “treatment” may refer generally to the effectuation of an operation controlled by the treatment planning system, such as the application of a beam or other optical input (e.g., radiation, acoustic, etc.) and “target”may refer to a non-anatomical object or area.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Reference throughout this specification to “one embodiment” or “an embodiment” means that a particular feature, structure, or characteristic described in connection with the embodiments included in at least one embodiment. Thus, the appearances of the phrase “in one embodiment” or “in an embodiment” in various places throughout this specification are not necessarily all referring to the same embodiment.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

What is claimed is:

1. A method of delivering radiotherapy, comprising:
generating a three-dimensional (3D) reference image of a patient;
generating a 3D image of a portion of the patient along a lengthwise axis;
performing a registration of the 3D image of portion of the patient to a corresponding portion of the 3D reference image; and
controlling a radiation treatment beam based on the registration.

2. The method of claim 1, wherein performing the registration comprises:
dividing the reference image into a plurality of volumes along the lengthwise axis; and locally registering the 3D image of the portion of the patient along the lengthwise axis to the corresponding volume of the plurality of volumes along the lengthwise axis.

3. The method of claim 1, further comprising:
generating a plurality of 3D images for a plurality of partial volumes of the patient during treatment; and
performing a registration of each of the plurality of 3D images to corresponding volumes of the 3D reference image.

4. The method of claim 3, wherein performing the registration of the plurality of 3D images of partial volumes of the patient provides a spatially dependent registration of the patient to the 3D reference image.

5. The method of claim 3, wherein each of the plurality of partial volumes are co-planar and leading a treatment field of the radiation treatment beam.

6. The method of claim 1, wherein the 3D image of the portion of the patient along the lengthwise axis is generated during radiation treatment delivery.

7. The method of claim 1, wherein controlling the radiation treatment beam comprises compensating the radiation treatment beam via at least one of a plurality of leaves, a plurality of jaw shifts of a multi-leaf collimator, adjusting a position of a treatment couch, or adjusting a position of the patient on the treatment couch.

8. A radiation delivery system, comprising:
one or more imaging devices;
a radiation treatment source; and
a processing device, operatively coupled with the radiation treatment source and the one or more imaging devices, wherein the processing device is to:
generate, by the one or more imaging devices, a three-dimensional (3D) reference image of a patient;
generate, by the one or more imaging devices, a 3D image of a portion of the patient along a lengthwise axis;
perform a registration of the 3D image of the portion of the patient to a corresponding portion of the 3D reference image; and
control a radiation treatment beam of the radiation treatment source based on the registration.

9. The radiation delivery system of claim 8, wherein to perform the registration, the processing device is to:
divide the reference image into a plurality of volumes along the lengthwise axis; and
locally register the 3D image of the portion of the patient along the lengthwise axis to the corresponding volume of the plurality of volumes along the lengthwise axis.

10. The radiation delivery system of claim 8, wherein the processing device is further to:
generate a plurality of 3D images for a plurality of partial volumes of the patient during treatment; and
perform a registration of each of the plurality of 3D images to corresponding volumes of the 3D reference image.

11. The radiation delivery system of claim 10, wherein performing the registration of the plurality of 3D images of partial volumes of the patient provides a spatially dependent registration of the patient to the 3D reference image.

12. The radiation delivery system of claim 10, wherein each of the plurality of partial volumes are co-planar and leading a treatment field of the radiation treatment beam.

13. The radiation delivery system of claim 8, wherein the 3D image of the portion of the patient along the lengthwise axis is generated during radiation treatment delivery.

14. The radiation delivery system of claim 8, wherein controlling the radiation treatment beam comprises compensating the radiation treatment beam via at least one of a plurality of leaves of a multi-leaf collimator, a plurality of jaw shifts of the multi-leaf collimator, adjusting a position of a treatment couch, or adjusting a position of the patient on the treatment couch.

15. A non-transitory computer-readable storage medium including instructions which, when executed by a processing device, cause the processing device to:
generate a three-dimensional (3D) reference image of a patient;
generate a 3D image of a portion of the patient along a lengthwise axis;
perform, by the processing device, a registration of the 3D image of the portion of the patient to a corresponding portion of the 3D reference image; and
control a radiation treatment beam based on the registration.

16. The non-transitory computer-readable storage medium of claim 15, wherein to perform the registration, the processing device is to:
divide the reference image into a plurality of volumes along the lengthwise axis; and
locally register the 3D image of the portion of the patient along the lengthwise axis to the corresponding volume of the plurality of volumes along the lengthwise axis.

17. The non-transitory computer-readable storage medium of claim 15, wherein the processing device is further to:
generate a plurality of 3D images for a plurality of partial volumes of the patient during treatment; and
perform a registration of each of the plurality of 3D images to corresponding volumes of the 3D reference image.

18. The non-transitory computer-readable storage medium of claim 17, wherein performing the registration of the plurality of 3D images of partial volumes of the patient provides a spatially dependent registration of the patient to the 3D reference image.

19. The non-transitory computer-readable storage medium of claim 17, wherein each of the plurality of partial volumes are co-planar and leading a treatment field of the radiation treatment beam.

20. The non-transitory computer-readable storage medium of claim 15, wherein the 3D image of the portion of the patient along the lengthwise axis is generated during radiation treatment delivery.
